(19) United States
(12) Patent Application Publication
(10) Pub. No.: US 2005/0245850 A1
(43) Pub. Date: Nov. 3, 2005

(54) Method and apparatus for inhibiting the growth of and shrinking cancerous tumors

(76) Inventors: Carlos V. Freyre, Miami, FL (US); Roger K. Khouri, Key Biscayne, FL (US)

Correspondence Address: THOMPSON COBURN, LLP, ONE US BANK PLAZA, SUITE 3500, ST LOUIS, MO, 63101, US

(21) Appl. No.: 11/101,240

(22) Filed: Apr. 7, 2005

Related U.S. Application Data
(63) Continuation-in-part of application No. 09/818,812, filed on Mar. 27, 2001.
Application No. 09/818,812 is a continuation of application No. 09/141,460, filed on Aug. 27, 1998, now Pat. No. 6,500,112.
Application No. 09/141,460 is a continuation of application No. 08/698,941, filed on Aug. 16, 1996, now abandoned.
Application No. 08/698,941 is a continuation-in-part of application No. 08/516,623, filed on Aug. 18, 1995, now Pat. No. 5,676,634.
Application No. 08/516,623 is a continuation-in-part of application No. 08/504,640, filed on Jul. 20, 1995, now Pat. No. 5,695,445.
Application No. 08/504,640 is a continuation of application No. 08/220,186, filed on Mar. 30, 1994, now Pat. No. 5,536,233.

(51) Int. Cl. A61B 19/00; A61H 7/00; A61F 5/00

(52) U.S. Cl. 601/6; 128/898

(57) ABSTRACT

A dome for applying a vacuum to a patient's breast is comprised of a generally rigid dome capable of withstanding a pressure differential, with a rim and rim cushion underlying the rim of the dome for supporting a rim from the patient's skin surface. The rim may be generally wider than the dome in order to distribute the attendant forces across a greater surface and avoid tissue damage. A sticky sole underlies the rim cushion and seals the rim cushion to the patient's skin to thereby preserve the vacuum within the dome. The sticky sole may be comprised of any adhesive material or even be achieved through the use of an appropriate material for the rim cushion itself. A portable pump unit is connected to the domes and maintains the vacuum within the domes during a recommended protocol. By using the device in accordance with the prescribed protocol, the growth of cancer cells and tumors within the breasts are inhibited.

This application is a continuation-in-part of co-pending U.S. patent application Ser. No. 09/818,812 filed Mar. 27, 2001, which is a continuation of co-pending U.S. patent application Ser. No. 09/141,460 filed Aug. 27, 1998 entitled “Vacuum Dome With Supporting Rim And Rim Cushion,” which is a continuation of U.S. patent application Ser. No. 08/698,941 filed Aug. 16, 1996 entitled “Vacuum Dome With Supporting Rim And Rim Cushion” (now abandoned), which is a continuation-in-part of U.S. patent application Ser. No. 08/516,623 filed Aug. 18, 1995 entitled “Method And Apparatus For Soft Tissue Enlargement With Balanced Force Appliance” (now U.S. Pat. No. 5,676,634), which is a continuation-in-part of U.S. patent application Ser. No. 08/504,640 filed Jul. 20, 1995 entitled “Method And Apparatus For Soft Tissue Enlargement By Distractive Force” (now U.S. Pat. No. 5,695,445), which is a continuation of U.S. patent application Ser. No. 08/220,186 filed Mar. 30, 1994 entitled “Method And Apparatus For Soft Tissue Enlargement” (now U.S. Pat. No. 5,536,233), the disclosures of all of the foregoing are hereby incorporated by reference.

### BACKGROUND AND SUMMARY OF THE INVENTION

There are numerous instances where persons desire enlargement of the soft tissues in their bodies. One such instance is for the replacement of one or both breasts amputated during a mastectomy in order to restore physiological symmetry and psychological well-being. Other instances are for correction of natural abnormalities such as dimpling. Still other instances are for augmentation of physical attributes to improve cosmetics and self-esteem. These latter soft tissue enlargements are principally directed to breast enlargement in females.

Prosthetic implants have been developed for insertion below the skin. However, the severity of the potential complications including scarring, implant rupture, capsular contracture, necrosis and implant migration as well as the recent adverse publicity thereof have significantly reduced the desirability of these implants. Thus, there is a societal need for other means to obtain soft tissue enlargement.

Some soft tissue enlargements occur naturally. For instance, during pregnancy, the skin over a woman's abdominal region enlarges approximately nine times its previous area to accommodate the fetus without a proportional decrease in skin thickness. In other words, the abdominal skin tissue actually enlarges and does not merely stretch during pregnancy. Similarly, the skin will expand to accommodate any growth under the skin.

In the past, plastic surgeons have used this phenomenon to their advantage to expand skin in order to accommodate prosthetic implants. To conduct this procedure, the surgeon inserts a balloon beneath the skin in the area where additional skin is desired. By progressively expanding the balloon, the skin first stretches and eventually actually grows to accommodate the increased volume underneath it. When the desired amount of skin is formed, the balloon is deflated and removed, and the implant is inserted into the cavity left by the balloon. Similar methods have been used by native African tribes to enlarge lips, nostrils, and earlobes.

Other surgical techniques have used tissue expansion to achieve other types of soft tissue growth. For instance, balloons have been successfully expanded underneath nerves, veins, tendons, and the like to thereby elongate these tissues to repair damage and alleviate various abnormalities.

A more advanced surgical method is known as callotasis or limb lengthening. This method comprises cutting the bone about its periphery at the location where lengthening is desired, leaving the tissues inside and around the bone intact. Brackets are attached to the bone on each side of the separation, and the bone segments are slowly pulled away from one another while remaining integral over a period of several months. Not only does this cause the mended bone to be longer, but also the soft tissue surrounding the bone actually grows to accommodate the increased limb length. Similar methods have been used by African native tribes to lengthen necks for cosmetic purposes.

Each of these above-mentioned apparatuses and methods requires an invasive surgical technique to accomplish the soft tissue expansion. Invasive techniques increase the likelihood of the complications associated with the procedure including those mentioned above with respect to implant surgery. In addition, the expense of surgery precludes many persons from having their abnormalities corrected or physical attributes enhanced.

Other soft tissue enlargement techniques have been developed which use other mechanisms to cause the enlargement. For instance, an instrument and technique have been developed for the non-surgical correction of inverted nipples due to short lactiferous ducts. The instrument is comprised of a cup having an internal volume shaped like that of the final desired nipple. The user places the cup over the inverted nipple, pumps the air out of the cup with a syringe and adjusts the vacuum within the cup using a check valve to just below the threshold of discomfort. Thus attached, the device puts the lactiferous ducts in tension and extends them sufficiently after two to three months of wear at 8-12 hours per day.

Although this device is sufficient for its intended purpose, it is not suitable for general soft tissue enlargement. Laceration and contusion can occur if too strong of a suction is applied to soft tissue. As the pressure within the inverted nipple instrument is not regulated, contusion or laceration can occur. When a vacuum is developed within the cup of the instrument, an equal and opposite force is applied to the patient about the rim of the cup. Excessive contact forces against the patient can cause ulceration, laceration, and contusions. As the contact forces are not regulated in the nipple instrument, these further complications also can occur. In addition, general soft tissue enlargement is not feasible with the instrument due to the size and shape of the cup.

Another prior art device is disclosed in U.S. Pat. No. 936,434 as a device for enlarging a woman's breasts. This device included a pair of cups for placement on the breasts and a pump for exhausting the air from between the cups and breasts. However, this patent provides no teaching as to the pressures to be used, the potential danger to the skin tissues, or any suggestions as to how the device is to be retained in place during use. Apparently, the device is used in a clinical setting and is not suitable for long term wear such as for 8-10 hours. As the patent suggests that the vacuum acts to cause the veins and arteries to engorge, thereby nourishing the breasts, it is clear that the patentee is suggesting that the breast tissue actually expands through this expansion of blood vessels alone. This patent has been the subject of ridicule by at least one medical authority. See “An Anthology Of Plastic Surgery” edited by Harry Hayes, Jr., M.D., Section 6, “Quackery and Nostrums” pub. 1986 by Aspen Publishers, Rockville, Md.

Most of these prior art devices and methods have failed to achieve long term soft tissue enlargement while preventing damage to the soft tissue being enlarged, as well as surrounding tissue. As disclosed and claimed in the parent applications and patents noted above, the inventor herein has succeeded in designing and developing a new generalized method and apparatus for soft tissue enlargement which prevents damage to soft tissue. The apparatus used for this enlargement is comprised of a rigid, fluid-impervious dome having a rim about its periphery and a vacuum pump for reducing pressure within the dome. The rim has sufficient surface area such that the pressure applied to the patient by the rim is less than or equal to the negative pressure applied to the soft tissue under the dome. In the parent patent filings, one specific teaching to achieve this balanced force utilized a rim with substantially the same cross-sectional area as the normal area of the dome. Thus, as long as pressure within the dome is regulated to a limit below which medical complications will not occur, the opposing contact pressure against the patient is below this threshold as well. With this approach, damage is avoided not only to the soft tissue being enlarged, but the surrounding tissue as well. In the preferred embodiment of the apparatus, the vacuum pump has a self-contained power source. In addition, a pressure sensor and servomechanism control the pump such that the vacuum within the dome is maintained at a magnitude less than 35 mmHg. Variant embodiments may be configured to fit over and enlarge a human breast, or any other desired soft tissue area.

In still another patent issued to one of the present inventors entitled “Method And Apparatus For Promoting Soft Tissue Enlargement and Wound Healing”, U.S. Pat. No. 5,701,917, issued Dec. 30, 1997, the disclosure of which is incorporated by reference, an invention was disclosed which utilizes a rigid fluid-impervious dome having a rim about its periphery and a vacuum pump for reducing pressure to thereby apply a distracting force to the soft tissue isolated by and within the dome. The dome may be conveniently located over an open wound in order to promote healing of the wound by enlarging the soft tissue under the dome. As the soft tissue grows, it promotes healing of the wound through acceleration of the closing thereof by soft tissue growth. As wounds may be received by a patient to any part of his body, the co-inventor's prior disclosed and claimed invention includes the use of a dome over virtually any part of the human body.

In implementing these prior inventions, it was intended that it be capable of achieving its therapeutic effect without creating any long term tissue necrosis from use. In other words, a vacuum must be applied to the desired area to achieve the therapeutic effect for sufficient periods of time without applying too great a vacuum or contact pressure which will damage the underlying tissue. As considered from this generalized approach, one of ordinary skill in the art would understand the inventor's teaching to include the idea of providing a smaller vacuum pressure within the dome and balancing that smaller vacuum with a rim having a surface area less than the normal area of the dome, thereby creating a greater contact pressure which is still within acceptable limits. Still another approach which may very well provide a therapeutic effect would be to cycle the vacuum in the dome such that it is applied for periods of time at elevated levels and relaxed levels so that the rim might also have a cross-sectional area less than the normal area of the dome, but yet avoid creating any tissue necrosis. The cycling of the vacuum pressure in the dome could be readily achieved in an automatic manner by appropriately programming the vacuum pump and regulator. Therefore, the prior inventions should be understood as being limited only by the current medical understanding of the causative effects of pressure sores and other tissue damage by an applied pressure or vacuum.

It is well recognized in the medical literature that decubitus ulcers are caused by unrelieved external pressure that occludes blood flow and results in tissue necrosis. In recognition of this fact, these ulcers are called pressure sores. The average capillary pressure in human skin is around 15-20 mmHg. E. M. Landis, Micro-Injection Studies of Capillary Blood Pressure in Human Skin, 15 Heart 209-228, (1930). For convenience, 20 mmHg has been used to describe this pressure throughout the previous patent filings. However, it should be understood that pressures below 20 mmHg may also be used without departing from the scope of these inventions and that these lower pressures may provide additional margins in preventing damage to tissues. Therefore, the local application of an external pressure up to 20 mmHg will not collapse capillaries adjacent the location of the applied pressure and thus will not disturb the circulation. Therefore, local application of contact pressures less than or equal to 20 mmHg are well tolerated for prolonged periods of time. This tolerance has been confirmed by the inventor through use of a prototype which did not cause adverse effects after many hours of continuous use as long as the pressure under the rim remained below or around 20 mmHg.

Pressures greater than 20 mmHg has generally been found to occlude the capillaries and stop tissue perfusion. Tissues can tolerate short periods of ischemia, but if the pressure is continuous and perfusion is not restored within a relatively short period of time, tissue damage will ordinarily ensue. “The time factor is thus more important than pressure intensity”. A pressure of 100 mmHg will lead to pathologic changes after only two hours. See T. Hussain, An Experimental Study of Some Pressure Effects on Tissues, with Reference to the Bed-Sore Problem, 66 J. Path. Bact. 347-358, (1953).

The experimental results of additional investigators can be used to develop a safe time-pressure curve above which tissue damage will ensue. For instance, 20 mmHg is well tolerated for prolonged periods of time, but 40 mmHg will lead to tissue injury if the pressure is not relieved for 13 hours. The injury is more severe if the pressure is 60 mmHg, and even greater injury will result with a pressure of 100 mmHg after shorter periods of time. O. Lindan, Etiology of Decubitus Ulcers: An Experimental Study, 42 Arch. Phys. Med. Rehab. 774-783, (1961). Similarly, a pressure of 70 mmHg, if unrelieved, will lead to pathologic changes after 2 hours. However, if the pressure is intermittent, applied 5 minutes on, and 5 minutes off, there is no pathologic tissue changes. M. Kosiak, Etiology of Decubitus Ulcers, 42 Arch. Phys. Med. Rehab. 19-29, (1961).

These findings are consistent with the clinical testing of the prototype of the breast device. It was found that a continuous pressure under the rim of 40 mmHg could be tolerated for only one hour by healthy volunteers. After one hour, the volunteers started to complain of pain which is the warning sign of impending tissue damage. Higher pressures led to pain under the rim after even shorter periods of time. Lower pressures around 30 mmHg led to pain after 4 hours. However, if the pressure is allowed to cycle, that is if it is dropped down to 0-20 mmHg to allow the tissues to temporarily reperfuse for a few minutes, higher peak pressures can be tolerated. The higher the peak pressures, the shorter they are tolerated and the longer the low pressure part of the cycle needs to be to allow the tissues to recuperate.

Therefore, pressures under the rim greater than 20 mmHg can only be tolerated if there is a means to continuously cycle the pressure peaks on and off allowing for tissue re-perfusion during the off periods. The higher the peaks, the shorter the pressures are tolerated and the longer the period of low pressure recuperation needs to be. From the above noted experimental animal data and human study, the inventor has previously concluded that a nominal 20 mmHg is the highest pressure that can be safely tolerated under the rim on a prolonged basis. Higher pressures can only be applied intermittently, and then cycled down to less than 20 mmHg.

The method of use for the previous invention is comprised of the steps of attaching the dome to the location of desired enlargement, and creating a vacuum within the dome. In the continuous application method in which the vacuum is applied at pressures that can be withstood continuously, the vacuum was recommended to be maintained for a minimum of eight hours per day and tissue enlargement results were expected to be sufficient after several months.

As indicated by the summary of the medical literature given above, a vacuum dome may also be used in alternative methods in keeping within the scope of the inventor's concept. For example, the device might have a rim cross-sectional area substantially less than the normal area of the dome and be used in either of two methods. In a first method, a somewhat lower vacuum pressure may be induced in the dome such that the opposing contact pressure under the rim may be maintained at bearable pressures for extended periods of time and yet provide a therapeutic effect. Alternatively, the vacuum in the dome may be regulated in a routine which provides somewhat higher vacuum pressures in the dome for shortened periods of time separated by periods of lower vacuum pressures to allow tissue reperfusion. In other words, alternating cycles of high vacuum, tissue reperfusion, high vacuum, tissue reperfusion, etc., may achieve a therapeutic effect in enlarging the soft tissues. With either of these methods, the rim may have a cross-sectional area substantially less than the normal area of the dome.

Alternate embodiments of dome construction may be found in other prior issued patents as noted above.

In implementing any of the embodiments of these prior inventions, the inventor utilizes a dome which is positioned adjacent a skin surface and which requires an airtight seal between the dome and the skin surface. In several of these embodiments, a vacuum may be drawn within the dome as well. In utilizing this construction, the inventor is aware of potential complications which can develop when an area of the body needs to be enclosed for prolonged periods of time within the dome having an airtight seal. For example, while a rim made of conforming or other soft materials may suffice for temporary use, a number of problems arise in the skin contact area when prolonged negative pressure application is necessary. The inventor has previously developed an invention which included in its various aspects various features which are intended to deal with these problems. These may be found in his prior issued US patents as noted above.

The inventor's device has been commercialized to great success over the last several years in a configuration disclosed and explained below as a preferred embodiment. As of year end 2004, the inventor's assignee has sold approximately 20,000 devices worldwide and 10,000 in the U.S. alone, comprising a portable bra embodiment for wear by women seeking the cosmetic advantages provided. As part of these sales, the buyers were provided the medical advice through medical professionals and data including their “wear” history was collected from approximately 78% of them. The recommended regimen required the user to wear the bra at least ten hours per day for ten consecutive weeks. Pressures exerted within the bra ranged within 15 to 33 mm Hg as the vacuum pump cycled off and on, with an average of approximately 22 mm Hg. Users were also advised that the more they wore the bra the better results they would achieve with an increased breast size. As a result, it is believed that many users chose to wear the device more than the ten hours per day and more than ten weeks. However, the recommended “wear” protocol was for ten hours per day for ten full weeks. As in any recommended protocol, and especially one extending for a significant time period, adjustments were made upon patient request and upon patient initiated change without advice. For some users, requests were granted to increase the maximum pressure to 35 and even 37 mmHg.

As another part of the protocol, a breast exam, medical history precluding those who had sisters or mothers experiencing pre-menopausal breast cancer, and screening mammography was suggested to qualify the potential user as eligible for purchasing and using the bra. This pre-screening was thought to be needed to avoid any unintended, and unknown at the time, potential for perhaps exacerbating a pre-existing pre-cancerous or even cancerous tumor or other growth within a user's breasts. Thus, admittedly, the pool of users did not totally reflect the female population as a whole. Nevertheless, it has been statistically demonstrated that between about 13 and 18 selected from the group of approximately 7700 users who contributed data would have been expected to contract detectable cancerous growth in their breasts over the course of the past several years.

Based on the unreported incidences of breast cancer, the statistical analysis of the data collected from the patient population, and the data expected to be collected from the patient population should further efforts be made, the inventors herein have conceived that the present invention evidences a method and apparatus for preventing the contraction of breast cancer. Having thus conceived of the invention, the inventors have investigated further and have found support in the medical literature for the medical efficacy of the method.

Mechanical forces are protean and omnipresent and have never been implicated as carcinogens. In the entire cancer research literature, there is no evidence that mechanical forces are remotely, theoretically or in any way associated with cancer causation or growth. In fact, crossing tension, expansion, or mechanical forces with cancer in a literature search engine leads to publications on the therapeutic implications of tissue expansion in cancer reconstruction.

The experiment of nature itself supports the idea that a cancer preventative effect would be experienced. The vacuum applied to the breasts effects a distracting force which is equivalent to the amount of gravity exerted from 4 pound breasts on a large-breasted women. Yet extensive epidemiological research confirms that women with larger breasts that are constantly subjected to the stretching effect of gravity are not at higher cancer risk than small-breasted women. In fact per gram of tissue, large breasts have been shown to have less cancer incidence than small ones.

Clinical experiments also support this result. Tissue expansion is routinely used for post-mastectomy breast reconstruction. It is well known that even the most radical mastectomy leaves behind some breast tissue. Yet more than three decades of experience on millions of women leave no evidence that expanding that residual cancer prone breast increases the likelihood of cancer growth or recurrence. Furthermore, despite the fact that skin is the most cancer prone organ in the body and skin expansion is routinely used in plastic surgery, there are no reports in the literature of cancer arising in the expanded skin.

Animal experiments also are supportive. Published research in animals of the effect of tissue expansion on cancer growth has shown that tissue expansion has an inhibitory effect upon the growth of implanted mammary tumor cells and on the extent of visceral metastasis. (Ref: Plast Reconstr Surg 1991 January; 87(1):1-7). These reported data support the notion that applying a vacuum not only prevents, but also may be used to therapeutically treat breast cancer.

Cell biology experiments suggest a physiological mechanism which might help explain how the invention works. The cellular mechanisms responsible for tension induced tissue growth involve a highly organized array of sensors, receptors and growth promoting signals. Transformed or cancerous cells typically lose these pathways and acquire an autonomous growth mechanism that is independent of these normal cues. When grown in tissue culture plates, normal cells multiply until they reach confluence; that is when a monolayer fills the plate and “contact inhibition” causes them to cease multiplying. Tissue expansion has been demonstrated in these tissue culture plates; as stretching the plate and the overlying cells causes them to multiply again until they reach the new equilibrium level of confluence. The hallmark of cancerous transformation is the loss of this “contact inhibition”. Cancer cells lose the normal clues that make them respond to confluence and tension and will keep on growing autonomously and independently from any additional stretch. In addition to the theoretical cell biology evidence (Stretching is good for a cell; Science: May 30, 1997; 276(5317):1345-6), there is evidence from an epidemiological study that showed that women who wear supportive brassieres that annul the lifelong stretch of gravity have a higher incidence of breast cancer. There is a body of medical literature that suggests that stretch is good for cell.

After four years of clinical practice in the US on thousands of women, the inventors can now affirm that the commercialized product has saved lives. There are a number of confirmed instances where the diligent requirement for breast exam and screening mammography has led to the detection and early treatment of smaller curable tumors that would have otherwise grown unnoticed and metastasized. As a rule, the inventors suggest that all users follow the guidelines of the American Cancer Society with regard to breast exam prior to using the invention. Expert opinions have been obtained from two nationally recognized breast cancer experts to independently review the data contained herein, the breast biopsies, and the literature and provide a written opinion as to the cancer risk, if any, associated with use of the invention prior to its commercialization. They both unequivocally opined that there is no evidence whatsoever that its use as recommended might in any way promote cancer growth or increase cancer risk. Studies have been conducted that also suggests that women who exercise have a lower incidence of breast cancer; and there is a Canadian study suggesting that older men that masturbate often have a lower incidence of prostate cancer.

The inventors have not yet definitively determined the physiological mechanisms which achieve this result. However, it is thought that as applying a vacuum or distractive force results in an increased perfusion of the breast, this increased perfusion may be a contributing factor as it:

a. Moves free radicals in/out faster cleansing the area,

b. Improves lymphatic filtration,

c. Enhances specific tissue growth factor,

d. Enhances growth factors that grow healthier tissue, and

e. Promote new fibroblast growth.

While the practical advantages and features of the present invention and method have been briefly described above, a greater understanding of the novel and unique features of the invention may be obtained by referring to the drawings and Detailed Description of the Preferred Embodiment which follow.

### BRIEF DESCRIPTION OF THE DRAWINGS

FIG. 1 is a front elevation view of the soft tissue enlargement apparatus, showing the breast augmentation embodiment;

FIG. 2 is a cross-sectional view of the breast enlargement embodiment taken in the plane of line 2-2 of FIG. 1;

FIG. 3 is a cross-sectional schematic of a dome and soft tissue in the early stages of enlargement;

FIG. 4 is a cross-sectional schematic of a dome and soft tissue in the latter stages of enlargement;

FIG. 5 is a partial cross-sectional view of the rim and rim cushion partially deflected to accommodate a bony prominence;

FIGS. 6A and 6B are cross-sectional views of the dome and rim with the rim cushions deflected to accommodate changes in the contour of the body surface;

FIG. 7 is a partial cross-sectional view of the dome and rim explaining the shear forces created at the rim;

FIG. 8 is a prospective view of a breast enlargement bra utilizing vacuum domes with a surrounding adhesive-coated bra;

FIG. 9 is a partial cross-sectional view of the bra depicted in FIG. 15 and detailing the vacuum dome, cushioned rim, and surrounding adhesive-coated strap arrangement;

FIG. 10 is a perspective view of two vacuum domes coupled to a portable vacuum pump as worn by a patient; and

FIG. 11 is a perspective view of the vacuum domes of FIG. 10 with a cloth bra worn by the patient to help support them.

### DETAILED DESCRIPTION OF THE PREFERRED EMBODIMENT

One embodiment of the soft tissue enlargement apparatus 10 is generally comprised of a dome 12 having a rim 14 and a vacuum pump assembly 16 for creating a vacuum within the dome. Although the vacuum pump assembly 16 may be a separate hand-held pump in one variant embodiment, in one preferred embodiment the vacuum pump assembly 16 is a self-contained vacuum pump 20 with an independent power source 22, pressure sensor 24, and servomechanism 26 for driving, regulating and controlling the vacuum pump 20.

Regulation of the vacuum within the dome is essential to prevent contusions caused by rupturing capillaries adjacent the surface of the skin. Medical data suggest that these contusions will not occur if vacuum within the dome is maintained at less than about 20-25 mmHg, at least on a continuous basis. Thus, the vacuum pump 20 must be regulated to control the vacuum within the dome to within this limit presuming a protocol involving extended wearing at continuous pressure is followed. In addition, skin ulceration can occur if excessive contact pressures are applied thereto for any extended time period. Medical data suggest that a contact pressure less than 20 mmHg may be applied indefinitely without such ulceration. However, contusions may occur due to positive contact pressures upon the skin at continuous pressures above this ulceration limit.

As the soft tissue enlarges, the rate of enlargement increases due to a beneficial physical phenomenon. If the tissue only slightly protrudes into the dome as shown in FIG. 3 and as is typically the initial condition, then the surface area 30 under the dome is only slightly larger than the normal area 32 at the dome opening. Therefore, the vacuum pressure P₁ acts on a surface area 30 which approaches the minimal value of the normal area. As enlargement occurs, more tissue protrudes into the dome 12 as shown in FIG. 4 thereby providing more surface area 30 under the dome. Because the surface area 30 under the dome is larger, the area over which the vacuum pressure acts is larger. For a given pressure, the enlargement of the soft tissue is a function of the surface area. Therefore, the total rate of enlargement of the soft tissue increases as treatment continues because the surface area under the dome is ever increasing. In other words, with more tissue under the dome the tensile force Fₛ is greater (Fₛ=PAₛ) and the breast grows larger faster. This however has no effect on the opposing force, or for that matter the normal force, as the tensile force Fₛ is a vector which must always sum into the normal force. In still other words, a unit of surface area enlarges at a constant rate for any given pressure, but as the soft tissue surface area under the dome increases, there are more units of surface area increasing at the constant rate. Therefore, the total rate of enlargement increases as treatment continues even though the vacuum pressure is not increased.

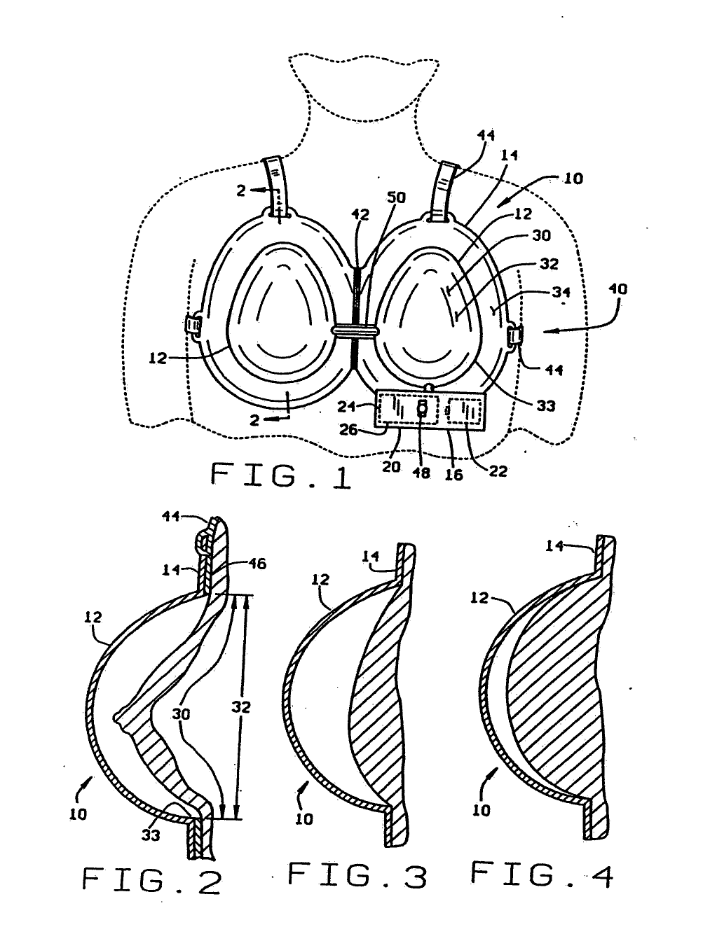

One specific embodiment includes a dome 12 configured to fit over a human breast as shown in FIGS. 1 and 2. This embodiment includes a rim 14 having a surface area 34 approximately equal to the normal area 32 of the dome opening thereby preventing medical complications to the soft tissue as long as the pressure is properly regulated within the dome 12. However, alternate embodiments having a rim 14 with a surface area 34 equal to or less than the normal area 32 of the dome opening may be used depending upon the amplitude of the vacuum pressure used and depending upon whether the vacuum pressure is constant or varied. The pressure reducing means 16 is located underneath the patient's breast, so that the apparatus 10 may be hidden under loose-fitting clothes. As with the general embodiment, the vacuum pump assembly 16 of this embodiment is preferably comprised of a vacuum pump 20 with a power source 22, a pressure sensor 24 and servomechanism 26 to drive and control the vacuum pump and to regulate the pressure within the dome 12.

As shown in FIG. 1, this specific embodiment may take the form of a bra 40 having two domes 12 spaced by a hinge 42. Straps 44 may be attached to the bra 40 to retain the bra 40 in place. A gasket 46 may also be included about the rim 14 to improve the patient's comfort and enhance the seal about the rim. In the preferred embodiment, this gasket 46 may be a silicone gel cushion or other soft, conforming type material. Petroleum jelly may also be used to supplement or supplant the gasket. A manual override 48 is included on the vacuum pump assembly 16 so that the patient or doctor may vary the pressure below the optimal level so as to be more comfortable. Although two vacuum pump assemblies 16 may be used, one depending from each dome 12 so as to provide different pressures in the domes, the preferred embodiment places the domes in fluid communication with a conduit 50. Two pump assemblies 16 may be desired to balance the size of two breasts as they are enlarged, as many women have differently sized breasts. Further, the pump may be replaced with a manually actuated pump such as a bulb-type pump.

In each of the above-described embodiments, the gasket 46 attached to the rim 14 may be configured to distribute any shear forces generated between the skin and rim as the tensile force is applied. This shear force distribution may be accomplished with the use of a silicone gel or inflated membrane or bladder which has a thickness sufficient to allow its surface 70 adjacent the soft tissue to shift laterally with respect to the rim. In this way, the shearing force is distributed along the surface 70 adjacent the soft tissue so that the force is not concentrated at the edge 72 of the rim adjacent the dome. In addition to distributing the shear forces over a larger area, the gel or other flexible rim material provides a cushion to improve the user's comfort and inhibit contusions should an unintentional impact be applied to the dome.

As shown in FIG. 5, still another physical attribute desirably accommodated by the vacuum dome and rim includes potential points of pressure concentration caused by a rib or other bony prominence 76 underlying the skin surface. As depicted therein, the rim cushion 46 underlying rim 14 should be sufficiently flexible to avoid creating a point of pressure concentration which could contribute to causing pressure sores or the like. This flexibility may be achieved for the use of a fluid-like cushion, an air-filled fluid bladder, a gel-like fluid, or such other construction and materials as would be effective to distribute the pressure substantially uniformly across the skin surface underlying the rim cushion 46.

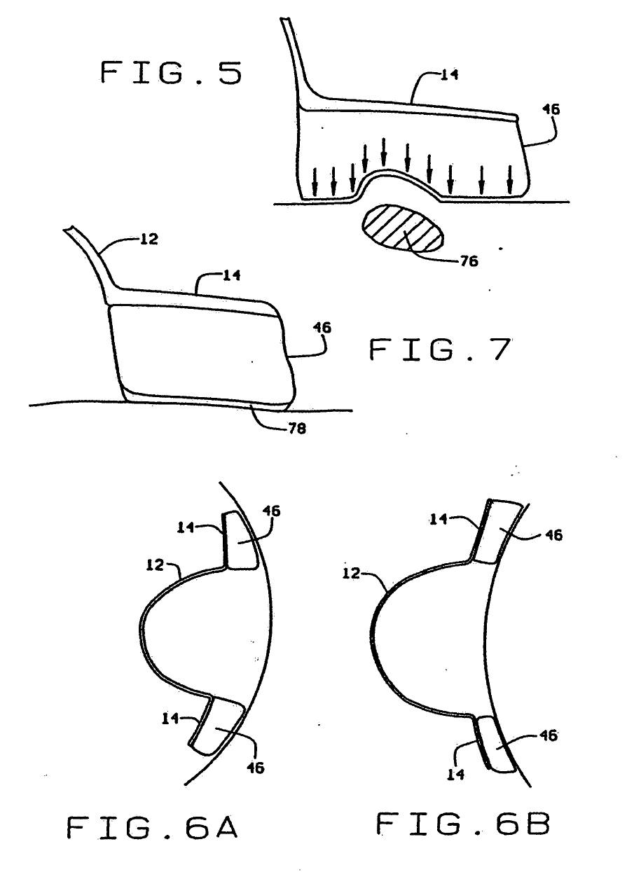

As shown in FIGS. 6A-B, the fluid-like cushion 46 described above, in some applications, should also accommodate an ever-changing contour of the skin surface as the user experiences his/her activities of daily living. This helps to avoid any potential vacuum loss from within dome 12 which would require re-establishing the vacuum. This helps to ensure reliable application of the vacuum to the intended skin surface without undo involvement with a pump. This ensures reliable results and minimal inconvenience to the patient.

As shown in FIG. 7, the inventors have also found it desirable to seal the rim cushion 46 to the skin surface through the use of a “sticky” sole interfaced between the rim cushion 46 and the skin surface. This “sticky” sole may be comprised of a number of alternative constructions. For example, the cushion 46 may itself be made of materials which exhibit a sufficiently “sticky” surface property so as to in and of itself provide this “sticky” function. Numerous polymers such as silicone, hydrogels, and many other low durometer synthetic rubbers and gels have this inherent surface property. Alternatively, another substance may be applied to the cushion 46, the underlying skin surface, or any combination thereof in order to achieve this “sticky” seal to ensure that the vacuum within dome 12 is reliably maintained as best as is feasible under the circumstances. This “sticky” sole 78 could also be a sheet or layer of an adhesive material, an adhesive layer may be applied to either the skin surface or rim cushion 46, a tape could be applied between the rim cushion 46 and skin surface, or some other such adhesive effect be achieved in any way which would be well known to those of ordinary skill in the art.

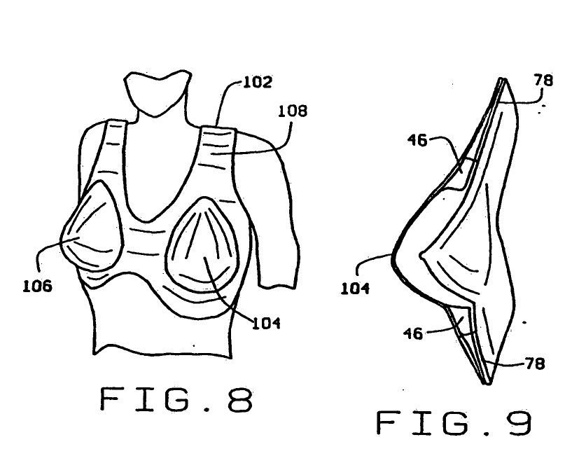

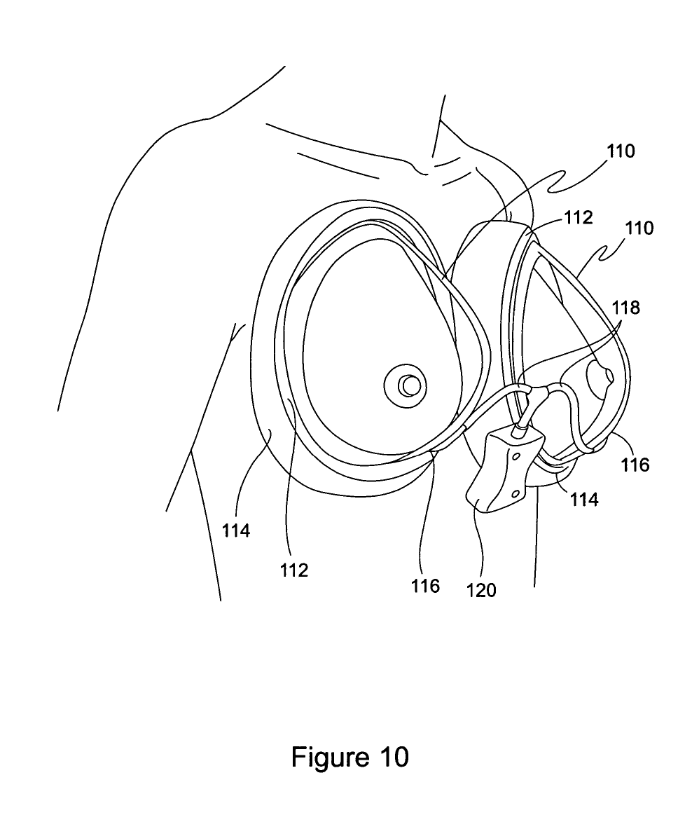

As shown in FIGS. 9 and 9, the “sticky” sole 78 need not necessarily underlie a rim cushion. As shown in FIG. 8, one of the intended embodiments of the inventor's vacuum dome includes a bra 102 including a pair of vacuum domes 104, 106 for increasing a woman's breast size. The sticky sole which provides the seal for the vacuum within vacuum domes 104, 106 may be applied between the straps 108 which surround the domes 104, 106 and, in effect, separated from the rim cushions 46. With this construction, the vacuum dome 104 and rim cushion 46 are mechanically separated from each other, although they should be joined to ensure the seal between the vacuum dome 104 and the underlying skin surface.

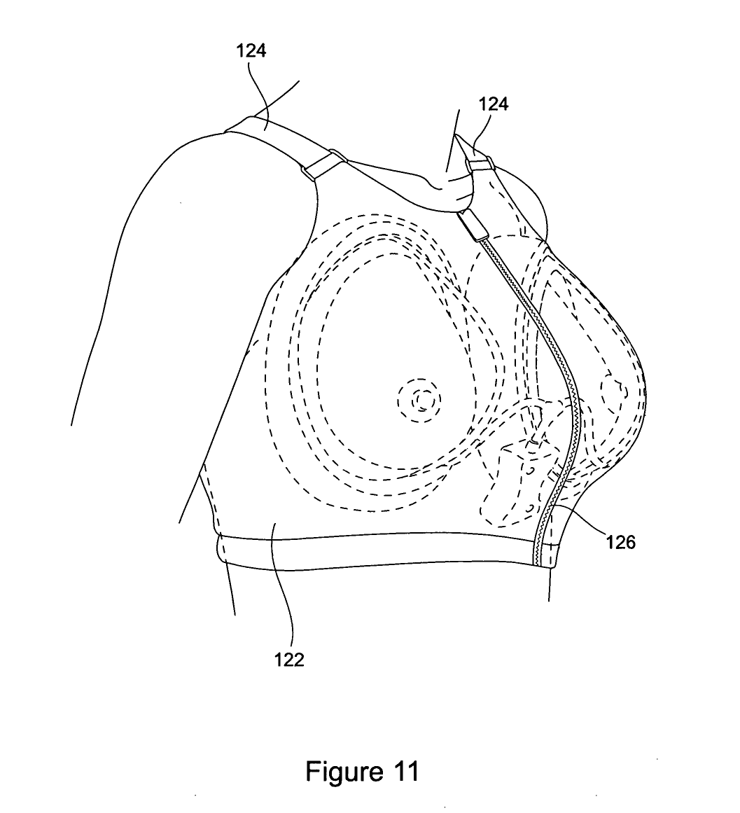

As shown in FIGS. 10 and 11, the commercialized version of the parent invention includes a pair of domes 110, each of which has a rim 112 and an underlying cushion 114 for supporting the domes from the patient's chest in a sealed condition. Each dome further has an opening 116 for attachment of a tube 118 leading to a junction and thence to a portable vacuum pump 120. A supporting cloth vest 122 as shown in FIG. 11 has adjustable shoulder straps 124 and a zippered front 126 for covering the domes 110 and holding them in place as the invention is worn. The portable pump 120 applied a pressure which cycled between about 15 to 33 mm Hg of pressure.

As mentioned above, commercial use of the invention in the United States by approximately 10,000 users over about 4 years, with data being collected from approximately 7700 users, has produced a single reported incidence of breast cancer which was deemed to have been pre-existing. The data is presented as follows.

Several assumptions were made using outside data sources that assimilate the patient data. Based on information published by the Surveillance, Epidemiology and End Results (SEER) Program, of the National Cancer Society, the incidence of females aged 20 to 54 years old being diagnosed with Breast Cancer is approximately 90 out 100,000 (or 0.09%) new cases per year. Factoring in the pre-selection criteria for buyers who never experienced breast cancer, or have sisters or mothers with a history of pre-menopausal breast cancer, the likelihood is reduced to 65 out 100,000 (or 0.065%) new cases per year. This assumed rate is based on information published by the Rhode Island Department of Health (HealthRI), which noted that approximately 18% of Breast Cancer is associated with some family history of infliction and the increased risk of approximately 2.5 times for those who have had a sister or mother who experienced Breast Cancer.

Based on the SEER and the HealthRI information, it is estimated that of the 7,689 units sold and registered by females between 2001 and Sep. 30, 2004, between about 13.4 and 18.6 cases of breast cancer would have been expected. See tables below.

Source: SEER Incidence of Breast Cancer by Age Distribution: for White US Females

TABLE 1
Summary Results at (i) 100%
Seers Incidence - Age Specific/White Females

| | Registered(*) | Breast Cancer By Age Group | | |
|---|---|---|---|---|
| Age | Brava Population | Expected 2001-2004(**) | | |
| Group | at Purchase | Invasive | In-Situ | Total |
| 15-19 | 211 | — | — | — |
| 20-24 | 918 | 0.02861 | — | 0.02861 |
| 25-29 | 1,191 | 0.22615 | — | 0.22615 |
| 30-34 | 1,474 | 0.95357 | 0.11088 | 1.06445 |
| 35-39 | 1,481 | 2.32314 | 0.43091 | 2.75405 |
| 40-44 | 1,231 | 3.91360 | 1.20000 | 5.11360 |
| 45-49 | 737 | 3.96103 | 1.18851 | 5.14954 |
| 50-54 | 306 | 2.16304 | 0.63917 | 2.80221 |
| 55-59 | 102 | 0.85229 | 0.22326 | 1.07555 |
| 60-64 | 26 | 0.22842 | 0.05158 | 0.28000 |
| 65-69 | 11 | 0.09312 | 0.02018 | 0.11330 |
| 70-74 | 1 | 0.01015 | 0.00197 | 0.01213 |
| 75-79 | — | — | — | — |
| 80-84 | — | — | — | — |
| Total | 7,689 | 14.75313 | 3.866449 | 18.61958 |

(*)Does not include purchases not registered.
(**)Data for 2004 ONLY Includes units sold and registered for the nine-months ended Sep. 30, 2004
(i) No adjustment for pre-selection criteria (i.e., exclusions for premenopausal breast cancer in immediate family, etc.)

TABLE 2
Summary Results at (ii) 72%
Seers Incidence - Age Specific/White Females

| | Registered(*) | Breast Cancer By Age Group | | |
|---|---|---|---|---|
| Age | Brava Population | Expected 2001-2004(**) | | |
| Group | at Purchase | Invasive | In-Situ | Total |
| 15-19 | 211 | — | — | — |
| 20-24 | 918 | 0.02060 | — | 0.02060 |
| 25-29 | 1,191 | 0.16283 | — | 0.16283 |
| 30-34 | 1,474 | 0.68657 | 0.07983 | 0.76640 |
| 35-39 | 1,481 | 1.67266 | 0.31025 | 1.98291 |
| 40-44 | 1,231 | 2.81779 | 0.86400 | 3.68179 |
| 45-49 | 737 | 2.85194 | 0.85572 | 3.70767 |
| 50-54 | 306 | 1.55739 | 0.46020 | 2.01759 |
| 55-59 | 102 | 0.61365 | 0.16075 | 0.77440 |
| 60-64 | 26 | 0.16447 | 0.03713 | 0.20160 |
| 65-69 | 11 | 0.06705 | 0.01453 | 0.08158 |
| 70-74 | 1 | 0.00731 | 0.00142 | 0.00873 |
| 75-79 | — | — | — | — |
| 80-84 | — | — | — | — |
| Total | 7,689 | 10.62225 | 2.78384328 | 13.40610 |

(*)Excludes people that purchased and did not register (approx. 2000 people)
(**)Data for 2004 ONLY includes units sold and registered for the nine-months ended Sep. 30, 2004
(i) No adjustment for pre-selection criteria.
(ii) Adjusted for pre-selection criteria by reducing incidence rates 28%.

While the particular protocol actually used by patients varied somewhat, the recommended protocol was for the patient to wear the device at least ten hours every day for ten weeks. This produced the results of not only growing or enlarging the soft tissue comprising the breasts, but also in completely eliminating any incidence of reported cancer over the time periods involved. As only a few years of data are presently available, the inventors are not yet scientifically certain as to whether the cancer inhibiting benefit accorded through use of the invention is permanent or time limited in some way. If time limited, it may very well be that repetition of the protocol would re-institute this cancer inhibiting effect such that a woman could be assured of being maximally protected from contracting breast cancer. This may only be learned through continued monitoring of the patient population to collect data that continues to indicate that no cancer has been contracted by any of the patients. The data available at the time of this filing includes some patients who used the invention approximately as long as 4 years ago. Thus the inventors feel confident that the cancer inhibiting effect lasts at least as long as 4 years.

It should also be noted that the data presented, and the conclusion that no cancer has been contracted in the patient population, may be verified through various means to lesser or greater statistical certainty. These include surveying cancer registers for patients names, surveying the medical professionals who prescribed and monitored the use of these devices for their individual experience with their patient population, selecting a random sampling of the overall patient population and then surveying that random sample including perhaps also having each patient surveyed submit to a mammogram, and a full patient population survey perhaps coupled with mammograms. One or more of these techniques could well be done with the statistical rigor necessary to produce a conclusion with a high degree of confidence confirming the experience of the assignee of the co-inventor's prior patents. However, in this litigious society, it is doubtful that a patient who was unfortunate enough to contract cancer would not provide notice to the assignee, perhaps through her attorney seeking compensation. Hence the inventors are confident that their conclusions will be borne out by any more rigorous testing of the data.

There are various changes and modifications which may be made to the invention as would be apparent to those skilled in the art. For example, while the preferred embodiments disclose generally the regimen of applying a vacuum to the patient, it is the desired effect of applying a distractive force to the underlying soft tissue that is believed to be important. Thus, this distractive force could be applied through other mechanical arrangements such as through the direct application of a physical or mechanical force or otherwise. This could be achieved without the need to create a vacuum. Still another alternative which is considered within the scope of the present invention is to modify the shape of the domes used to encapsulate the breasts. For convenience, the domes are described and depicted in the classic shape of a curvilinear solid. This shape follows the contour of the breast and is thought to be less noticeable when worn. However, other shapes could be used as well, and such shapes are to be considered as within the definition of the term “dome” as used herein. While a specific protocol for wearing the invention has been recommended, it is also indicated herein that there is room for variation in this recommended protocol and it should be understood that in the real world very few if any patients would have followed the recommended protocol exactly. For example, circumstances could well have limited the amount of time during any one day that the invention was worn. Still another variation could well be that the patient might have seen fit to skip one or more days due to various reasons and not worn the invention at all during that time. However, these changes or modifications are included in the teaching of the disclosure and it is intended that the invention be limited only by the scope of the claims appended hereto.

1. A method for inhibiting the growth of cancer cells in a patient's breast comprising applying a distractive force to said patient's breast in substantial compliance with a recommended protocol.

2. The method of claim 1 wherein applying the distractive force includes applying it with a device comprising a dome adapted to surround at least a portion of said patient's breast, said dome being configured for supporting a vacuum pressure between said patient's breast and said dome, the vacuum pressure thereby applying the distractive force to said patient's breast.

3. The method of claim 2 wherein said dome further comprises a rim for supporting said dome against said distractive force, said rim having a sticky surface adapted to adhere to the patient's skin to thereby create and hold a pressure seal between said dome and said patient.

4. The method of claim 1 wherein the patient is a human.

5. The method of claim 4 wherein the human is a woman.

6. The method of claim 1 wherein the cancer cells are benign.

7. The method of claim 1 wherein the cancer cells are malignant.

8. The method of claim 7 wherein the cancer cells are metastatic.

9. The method of claim 1 wherein performing said method substantially inhibits the growth of cancer cells for at least 4 years thereafter.

10. A method for reducing the risk of an incidence of breast cancer in a patient comprising applying a distractive force to said patient's breast in substantial compliance with a recommended protocol.

11. The method of claim 10 wherein applying the distractive force includes applying it with a device comprising a dome adapted to surround at least a portion of said patient's breast, said dome being configured for supporting a vacuum pressure between said patient's breast and said dome, the vacuum pressure thereby applying a distractive force to said patient's breast.

12. The method of claim 11 wherein said dome further comprises a rim for supporting said dome against said distractive force, said rim having a sticky surface adapted to adhere to the patient's skin to thereby create and hold a pressure seal between said dome and said patient.

13. The method of claim 10 wherein the patient is a human.

14. The method of claim 13 wherein the human is a woman.

15. The method of claim 10 wherein the cancer cells are benign.

16. The method of claim 10 wherein the cancer cells are malignant.

17. The method of claim 16 wherein the cancer cells are metastatic.

18. The method of claim 10 wherein performing said method substantially reduces the incidence of breast cancer for at least 4 years thereafter.

19. A device for inhibiting the growth of cancer cells in a patient's breast comprising at least one dome, said dome being configured to withstand the creation of a vacuum between said dome and a patient's breast, and a pump coupled to said dome wherein said pump when operated evacuates air from within said dome to thereby create a vacuum therein.

20. A device for reducing the risk of incidence of breast cancer in a patient comprising at least one dome, said dome being configured to withstand the creation of a vacuum between said dome and a patient's breast, and a pump coupled to said dome wherein said pump when operated evacuates air from within said dome to thereby create and maintain a vacuum therein.
